(19) United States
(12) Patent Application Publication
(10) Pub. No.: US 2020/0368578 A1
(43) Pub. Date: Nov. 26, 2020

(54) STEP-COUNTING TREADMILL

(71) Applicant: CHUN TA LIN, Taichung City (TW)

(72) Inventor: CHUN TA LIN, Taichung City (TW)

(21) Appl. No.: 16/419,090

(22) Filed: May 22, 2019

(51) Int. Cl. A63B 24/00; A63B 22/02; A61B 5/103; A61B 5/00

(52) U.S. Cl. CPC A63B 24/0062; A63B 22/025; A61B 5/1038; A63B 22/0285; A63B 2071/0658; A63B 2024/0093; A63B 2220/58; A63B 2220/833; A61B 5/4866

(57) ABSTRACT

A step-sensing treadmill includes a frame assembly includes a lower frame securely mounted on a ground, a controller electrically connected to a dashboard and two step-sensing units each of which is mounted between the walking belt assembly and a left side or a right side of the lower frame of the frame assembly, and is collaborated with the controller to continuously sense a voltage variation caused by user's movement on walking belt relative to the walking belt in a motionless state. When the first voltage variation is greater than a predetermined voltage variation threshold, the controller determines that one user's left or right step on the walking belt is made, and increments a left or right step number by one. The left and right step numbers can be used to further calculate calorie burned, and determine exercising conditions of the user during an exercise activity.

### BACKGROUND OF THE INVENTION

### 1. Field of the Invention

The present invention relates to a treadmill and, more particularly, to a step-counting treadmill with at least one step-counting unit mounted on one of a left side and a right side of a lower frame of the step-counting treadmill.

### 2. Description of the Related Art

Workout in an indoor gym is one of the most efficient choices available to people looking forward to getting fit and staying healthy especially to those active in urban areas or in areas where the likelihood of getting access to outdoor exercise appears to be low. Among all equipment in the gym, treadmill can be undoubtedly considered as the equipment that users can use for a straightforward longer run or walk works to burn calories.

Conventional treadmills calculate calories burned for users with the speed of the treadmill, the grade of the work, the weight of the user, and the time spent for exercising. However, all these factors involved for calculating the calories burned only allow the conventional treadmills to calculate the calories burned based on a statistical way which fails to take user's step movement into account and thus leads to the calculated calories burned not actually reflecting the physical work of the user done on the treadmill Besides, owing to no awareness of the user's step movement, the conventional treadmills can hardly be treated as a friendly exercise platform with the capability of reminding the user for any belt speed change when the user's steps do not keep up with the belt speed to a lesser extent, indicative of a situation that the grade selected is slightly above the user's physical strength in a uncomfortable but not yet painful range, and can also hardly be treated as a risk-free exercise platform with the capability of immediately stopping operation the conventional treadmills when the user's steps apparently fall behind the belt speed to a greater extent, indicative of a situation that the grade selected totally overwhelms the user's physical strength and may place the user in jeopardy during the workout.

### SUMMARY OF THE INVENTION

An objective of the present invention is to provide a step-counting treadmill capable of counting steps made by a user on a treadmill by utilizing at least one step sensor to sense a voltage variation as a result of a movement change caused by user on the treadmill and determine whether a step is made by the user according to the voltage variation.

To achieve the foregoing objective, the step-counting treadmill includes a frame assembly, a dashboard, a walking belt assembly, a controller, a drive motor, and a step-sensing unit.

The frame assembly has a lower frame and an upper frame.

The lower frame is securely mounted on a ground.

The upper frame is mounted on the lower frame.

The dashboard is mounted on the upper frame.

The walking belt assembly is mounted on the frame assembly.

The controller is mounted inside the frame assembly, is electrically connected to the dashboard, and initializes a total step number of an exercise activity of a user on the walking belt assembly to zero before the exercise activity starts.

The drive motor is electrically connected to the controller and is instructed by the controller to drive the walking belt assembly to move according to a treadmill speed inputted by the user through the dashboard for the exercise activity.

The step-sensing unit is electrically connected to the controller, is mounted between the walking belt assembly and a left side or a right side of the lower frame of the frame assembly, and is collaborated with the controller to continuously sense a voltage variation as a result of a movement change between a vibrated state of the walking belt assembly caused by a movement of the user on the walking belt assembly during the exercise activity and a motionless state of the walking belt assembly before the exercise activity starts.

When the voltage variation is greater than a predetermined voltage variation threshold, the controller is configured to determine that one step on the walking belt assembly is made by the user and increments the total step number by one.

As can be seen from the foregoing description, the step-sensing treadmill has only one step-sensing unit, the availability of a single step-sensing unit allows total step counting, regardless of left or right steps. Owing to the availability of the total step number, a user's calories burned for an exercise activity can thus be calculated. However, as the step-sensing treadmill with only one step-sensing unit is unable to distinguish the left steps from the right steps, other application of the step-sensing treadmill may include detection of an idle state occurs when there is no step detected for a while.

To achieve the foregoing objective, the step-counting treadmill includes a frame assembly, a dashboard, a walking belt assembly, a controller, a drive motor, and two step-sensing units.

The frame assembly has a lower frame and an upper frame.

The lower frame is securely mounted on a ground.

The upper frame is mounted on the lower frame.

The dashboard is mounted on the upper frame.

The walking belt assembly is mounted on the frame assembly.

The controller is mounted inside the frame assembly, is electrically connected to the dashboard, initializes a left step number, a right step number, a total step number, a step cycle, and an exercise duration of a user in an exercise activity to zero before the exercise activity starts, and starts timing the step cycle and the exercise duration since the exercise activity starts.

The drive motor is electrically connected to the controller and instructed by the controller to drive the walking belt assembly to move according to a treadmill speed inputted by the user through the dashboard.

The two step-sensing units are electrically connected to the controller. One of the step-sensing units is mounted between the walking belt assembly and a left side of the lower frame of the frame assembly, and is collaborated with the controller to continuously sense a first voltage variation as a result of a movement change between a vibrated state of the walking belt assembly caused by a movement of the user on the walking belt assembly during the exercise activity and a motionless state of the walking belt assembly before the exercise activity starts. the other step-sensing unit is mounted between the walking belt assembly and a right side of the lower frame of the frame assembly, and is collaborated with the controller to continuously sense a second voltage variation as a result of a movement change between a vibrated state of the walking belt assembly caused by a movement of the user on the walking belt assembly during the exercise activity and a motionless state of the walking belt assembly before the exercise activity starts.

When the first voltage variation is greater than a predetermined voltage variation threshold, the controller is configured to determine that one left step on the walking belt assembly is made by the user, increment the left step number by one, set the step cycle as a user's left step cycle, and re-initialize the step cycle to zero. When the second voltage variation is greater than the predetermined voltage variation threshold, the controller is configured to determine that one right step on the walking belt assembly is made by the user, increment the right step number by one, set the step cycle as a user's right step cycle, and re-initialize the step cycle to zero.

Compared with the foregoing step-counting treadmill with a single step-sensing unit, the step-counting treadmill with two step-sensing units in the above-mentioned description can being forth more applications, such as determination of the user's exercising condition on the walking belt based on comparisons between the user's left/right step cycle and the targeted left/right step cycle, simply because of the capability of sensing the left steps and the right steps.

Other objectives, advantages and novel features of the invention will become more apparent from the following detailed description when taken in conjunction with the accompanying drawings.

### BRIEF DESCRIPTION OF THE DRAWINGS

FIG. 1 is a schematic perspective view showing a step-counting treadmill with one step-sensing unit on the left of the treadmill in accordance with the present invention;

FIG. 2 is a schematic perspective view showing the step-counting treadmill with one step-counting unit on the right of the treadmill in accordance with the present invention;

FIG. 3 is a schematic perspective view showing the step-counting treadmill with two step-counting units on both sides of the treadmill in accordance with the present invention;

FIG. 4 is another schematic perspective view showing the step-counting treadmill in FIG. 1;

FIG. 5A is a schematic side view of a step-counting treadmill with a Hall sensor and a magnet on the left side of the treadmill in accordance with the present invention; and

FIG. 5B is an enlarged side view of the Hall sensor and the magnet of the step-counting treadmill in FIG. 5A.

### DETAILED DESCRIPTION OF THE INVENTION

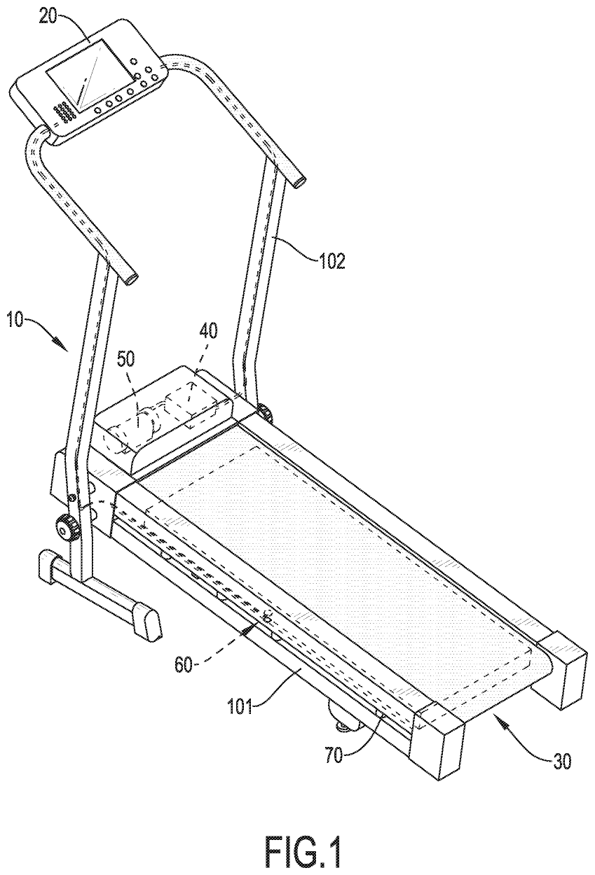

With reference to FIGS. 1 to 4, a step-counting treadmill in accordance with the present invention includes a frame assembly 10, a dashboard 20, a walking belt assembly 30, a controller 40, a drive motor 50, and at least one step-sensing unit 60. The frame assembly 10 has a lower frame 101 and an upper frame 102. The lower frame 101 is securely mounted on a ground to ensure that the treadmill is not jiggling and movable with respect to the ground. The upper frame 102 is mounted on the lower frame 101. The dashboard 20 is mounted on the lower frame 101 to serve as a console for receiving a user's inputs and instructions for an exercise activity desired by the user on the walking belt assembly 30 and displaying exercise information and status of the exercise activity. The walking belt assembly 30 is mounted on the frame assembly 10 and is dedicated for the user to run or walk thereon during the exercise activity. The controller 40 is mounted inside the frame assembly 10 and is electrically connected to the dashboard 20 through cabling. The drive motor 50 is electrically connected to the controller 40 through cabling and is instructed by the controller 40 to drive the walking belt assembly 30 to move according to a treadmill speed inputted by the user for the exercise activity through the dashboard 20 for the exercise activity. The at least one step-sensing unit 60 is electrically connected to the controller 40 through cabling. Each of the at least one step-sensing unit 60 is mounted between the walking belt assembly 30 and a left side or a right side of the lower frame 101 of the frame assembly 10, and is collaborated with the controller 40 to continuously sense a voltage variation as a result of a movement change between a vibrated state of the walking belt assembly 30 caused by a movement of the user on the walking belt assembly 30 in the exercise activity and a motionless state of the walking belt assembly 30 before the exercise activity starts.

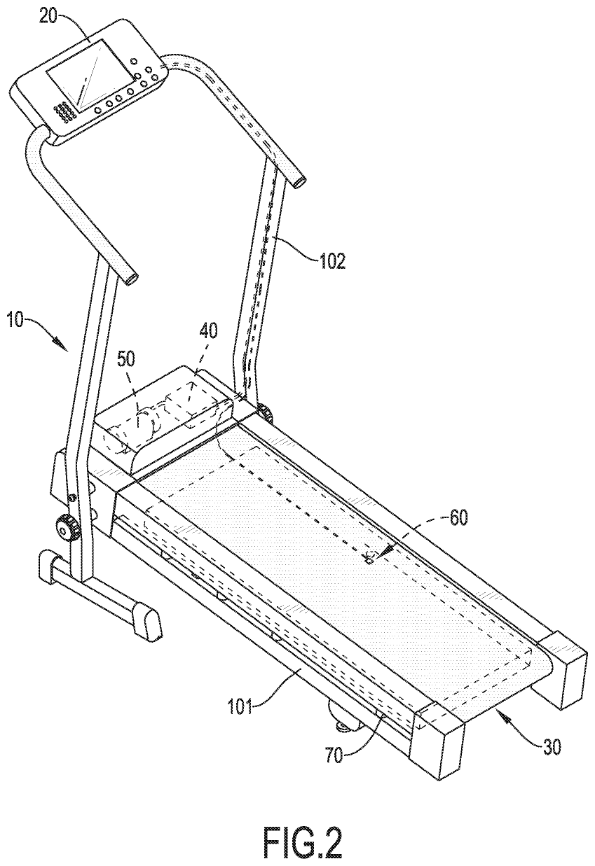

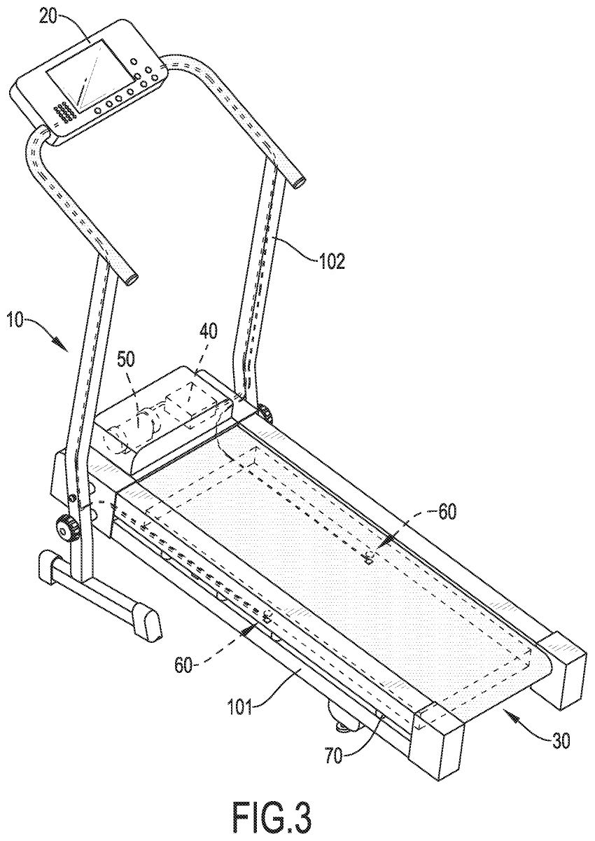

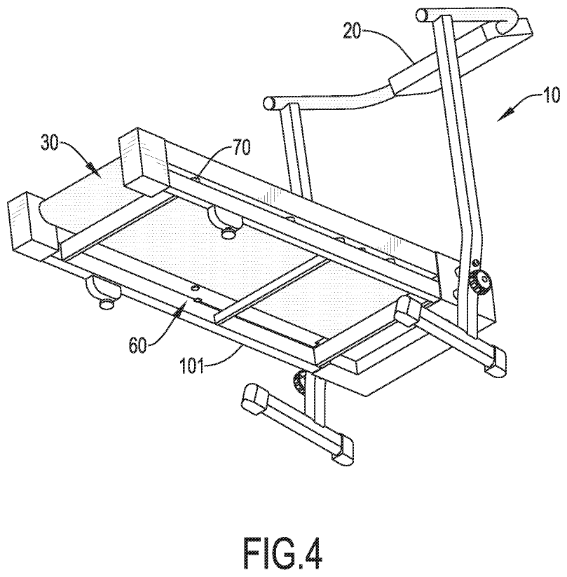

The at least one step-sensing unit 60 may include one or more step-sensing units 60. FIGS. 2 and 3 show the embodiments with only a single step-sensing unit 60, and the step-sensing unit 60 thereof is selectively mounted between the walking belt assembly 30 and the left side or the right side of the lower frame 101. FIG. 4 shows an upward view of the step-sensing unit 60 mounted between the left side of the lower frame 101 and the walking belt assembly 30. For purpose of calculating the total calories burned for the exercise activity, the controller 40 initializes a total step number to zero before the exercise activity starts. As the voltage variation is continuously sensed, when the voltage variation sensed by the single step-sensing unit 60 in FIG. 1 or FIG. 2 is non-zero and greater than a predetermined voltage variation threshold, the controller 40 determines that a step, regardless of a left step or a right step, is made by the user on the walking belt assembly 30 and increments the total step number by one. In the scenario, the step-sensing unit 60 is unaware of whether a left step or a right step is made. Each of an actual left step and an actual right step sensed by the single step-sensing unit 60 is treated as one step. However, to ensure accuracy of sensing a step on the walking belt assembly 30 that is opposite to a side on which the single step-sensing unit 60 is mounted, for example, a right step sensed by a right-sided step-sensing unit 60, the sensitivity for the single step-sensing unit 60 may need to be increased because the step opposite to the step-sensing unit 60 appears to be more distant than the step on the same side of the step-sensing unit 60. To address such sensitivity concern, the predetermined voltage variation threshold may be set to be relatively lower for the single-side step-sensing unit 60 to be able to sense a step on the opposite side of the step-sensing unit 60.

As the total step number is available from the step counting, the controller can use the total step number as one parameter for calculating calories burned during the exercise activity. Moreover, when determining that no step has been sensed for an idle period, the controller automatically stops operation of the walking belt assembly 30 and let the step-counting treadmill enter a hibernation state, and when determining that any step is sensed during the hibernation state, the controller then instructs the dashboard to display a message of reminding the user to resume the exercise activity.

Differing with FIGS. 1 and 2 in terms of the number of the at least one step-sensing unit 60, FIG. 3 shows the embodiment with two step-sensing units 60, one of which is mounted between the walking belt assembly 30 and a left side of the lower frame 101 of the frame assembly 10 and is collaborated with the controller 40 to continuously sense a first voltage variation as a result of a movement change between a vibrated state of the walking belt assembly 30 caused by a movement of the user on the walking belt assembly 30 in the exercise activity and the motionless state of the walking belt assembly 30 before the exercise activity starts, and the other step-sensing unit 60 which is mounted between the walking belt assembly 30 and a right side of the lower frame 101 of the frame assembly 10 and is collaborated with the controller to continuously sense a second voltage variation as a result of another movement change between another vibrated state of the walking belt assembly 30 caused by another movement of the user on the walking belt assembly 30 in the exercise activity and the motionless state of the walking belt assembly 30 before the exercise activity starts. To calculate the total calories burned and keep track statuses of left steps and right steps during the exercise activity of the user, the controller 40 initializes a left step number, a right step number, the total step number, a step cycle, and an exercise duration in the exercise activity to zero before the exercise activity starts, and starts timing the step cycle and the exercise duration since the exercise activity starts. When the first voltage variation is non-zero and greater than the predetermined voltage variation threshold, the controller 40 determines that one left step on the walking belt assembly 30 is made by the user, increments the left step number by one, sets the step cycle as a user's left step cycle, and re-initializes the step cycle to zero. When the second voltage variation is non-zero and greater than the predetermined voltage variation threshold, the controller 40 determines that one right step on the walking belt assembly 30 is made by the user, increments the right step number by one, sets the step cycle as a user's right step cycle, and re-initializes the step cycle to zero.

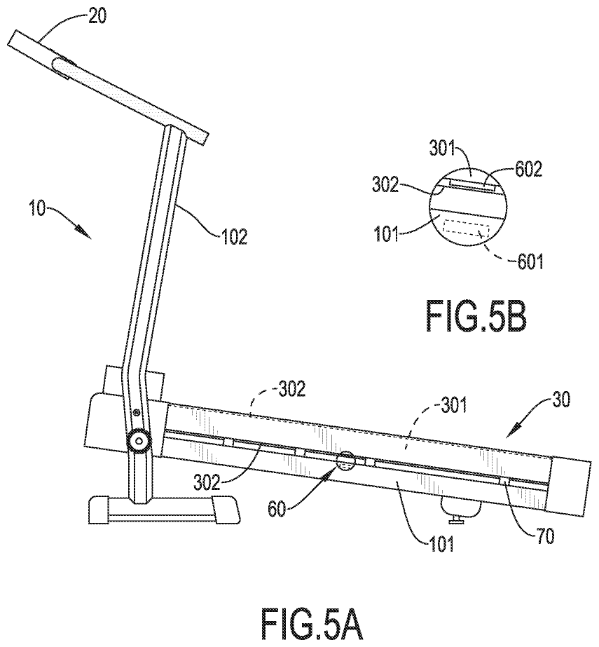

With reference to FIGS. 5A and 5B, the walking belt assembly 30 includes a roller bracket 301 and a walking belt 302. The roller bracket 301 is mounted above the lower frame 101 with cushioning pieces 70 sandwiched between the roller bracket 301 and the lower frame 101. The walking belt 302 is movably mounted around the roller bracket 301 and is driven by the drive motor 50 to move around the roller bracket at the treadmill speed instructed by the controller 40. In the foregoing embodiments, each of the at least one step-sensing unit 60 may include a Hall sensor 601 and a magnet 602. The Hall sensor 601 is mounted on a left side or a right side of the lower frame 101 and is electrically connected to the controller 40. The magnet 602 is mounted on the roller bracket 301 to contactlessly correspond to the Hall sensor 601 in position. The Hall sensor 601 senses a first magnitude of a magnetic field of the magnet 602 when the magnet 602 is in a motionless state before the exercise activity starts and converts the first magnitude into a first voltage value. The Hall sensor 601 further senses a second magnitude of the magnetic field of the magnet 602 when the magnet 602 is in a vibrated state as a result of a movement of the user on the walking belt assembly 30 in the exercise activity and converts the second magnitude into a second voltage value. The controller 40 then calculates a voltage variation by subtracting the second voltage value from the first voltage value. When the voltage variation is non-zero and is greater than a predetermined voltage variation threshold, the controller 40 determines that one step on the walking belt assembly is made by the user and increments the total step number by one. The Hall sensor 601 and the magnet 602 may include but are not limited to the above-mentioned mounting locations as long as the resultant sensing effect is acceptable.

In view of the availability of the left step number, the right step number, the left step cycle and the right step cycle, besides calculation of the calories burned for the exercise activity and detection of the idle state, the embodiment with two step-sensing units 60 in FIG. 4 is more versatile than those in FIGS. 2 and 3 in additionally identifying a slow step condition, a dangerous step condition of the user. Firstly, speaking of calculation of the calories burned for the exercise activity, the controller 40 calculates the total step number by adding the left step number and the right step number and uses the total step number as one parameter for calculating calories burned for the exercise activity. As for detection of the idle state, when determining that none of the left step and the right step has been sensed for an idle period, the controller 40 automatically stops running the walking belt and enters a hibernation state, and when determining that any left step or any right step is sensed during the hibernation state, the controller 40 instructs the dashboard 20 to display a message reminding the user to resume the exercise activity.

As for how to identify the slow step condition, with a height of the user inputted through the dashboard, the controller 40 may calculate a targeted left step cycle and a targeted right step cycle according to the treadmill speed, the exercise duration of the exercise activity, and the height of the user, and determines that a slow step condition of the user occurs when a difference between the targeted left step cycle and the user's left step cycle at present is greater than a lower targeted gap and is less than an upper targeted gap or a difference between the targeted right step cycle and the user's right step cycle at present is greater than the lower targeted gap and is less than an upper targeted gap. When determining that the user is walking in a slow step condition, the controller 40 instructs the dashboard 20 to display a message reminding the user to speed up his/her walking speed and asking him/her whether the treadmill speed should be reduced.

As for how to identify the dangerous step condition, the controller 40 determines that a dangerous step condition of the user occurs when a difference between the targeted left step cycle and the user's left step cycle at present is greater than the upper targeted gap or a difference between the targeted right step cycle and the user's right step cycle at present is greater than the upper targeted gap. When determining that the user is running in a dangerous step condition, the controller 40 instructs the dashboard 20 to display a message reminding the user of his/her running speed falling far behind the treadmill speed and automatically stops the operation of the walking belt assembly 30.

As for how to identify the inconsistent step condition, the controller 40 determines that an inconsistent step condition of the user occurs when an absolute value of a difference between the user's left step cycle and the user's right step cycle at present exceeds an inter-step cycle variation. When determining that the user is walking/running in an inconsistent step condition, the controller 40 instructs the dashboard 20 to display a message reminding the user of his/her walking or running in an inconsistent step pace that requires his/her attention for step adjustment.

In sum, the step-sensing treadmill in accordance with the present invention utilizes the at least one step-sensing unit to sense a change in an external magnetic field caused by vibration of an object to be sensed mounted on the walking belt assembly as a result of user's exercise activity and convert that change in magnetic field into a voltage value of the object in motion. A voltage variation between the voltage value and a sensed voltage value of the object in a motionless state can be employed to determine whether there is a step made by the user on the walking belt. When there is only one step-sensing unit mounted on the step-sensing treadmill, a total calories burned for the user's exercise activity can be calculated and detection of an idle state into which the user is running can be implemented. In contrast to the treadmill with a single step-sensing unit, besides the functions available to the case with single step-sensing unit, the treadmill with two step-sensing units can further detect the slow step condition, the dangerous step condition and the inconsistency step condition according to comparisons between the user's step cycles and the targeted step cycles for both the left steps and the right steps. Furthermore, when each of the at least one step-sensing unit includes a Hall sensor and a magnet contactlessly corresponding to each other in position, the Hall sensor and the magnet as a whole deliver the advantages of high reliability and easy mounting as being contactless to each other, energy saving, compact in size, and good sensitivity.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A step-counting treadmill comprising:
a frame assembly having:
a lower frame securely mounted on a ground; and
an upper frame mounted on the lower frame;
a dashboard mounted on the upper frame;
a walking belt assembly mounted on the frame assembly;
a controller mounted inside the frame assembly, electrically connected to the dashboard, and initializing a total step number of an exercise activity of a user on the walking belt assembly to zero before the exercise activity starts;
a drive motor electrically connected to the controller and instructed by the controller to drive the walking belt assembly to move according to a treadmill speed inputted by the user through the dashboard for the exercise activity; and
a step-sensing unit electrically connected to the controller, mounted between the walking belt assembly and a left side or a right side of the lower frame of the frame assembly, and collaborated with the controller to continuously sense a voltage variation as a result of a movement change between a vibrated state of the walking belt assembly caused by a movement of the user on the walking belt assembly during the exercise activity and a motionless state of the walking belt assembly before the exercise activity starts;
wherein when the voltage variation is greater than a predetermined voltage variation threshold, the controller is configured to determine that one step on the walking belt assembly is made by the user and increments the total step number by one.

2. The step-counting treadmill as claimed in claim 1, wherein
the walking belt assembly includes:
a roller bracket mounted above the lower frame with cushioning pieces sandwiched between the roller bracket and the lower frame; and
a walking belt movably mounted around the roller bracket and driven by the drive motor to move around the roller bracket at the treadmill speed instructed by the controller;
the step-sensing unit has:
a Hall sensor selectively mounted on a left side or a right side of the lower frame and electrically connected to the controller; and
a magnet mounted on the roller bracket to contactlessly correspond to the Hall sensor in position, wherein the Hall sensor senses a first magnitude of a magnetic field of the magnet when the magnet is in a motionless state and converts the first magnitude into a first voltage value, a movement of the user on the walking belt assembly causes the magnet mounted on the roller bracket to vibrate, and the Hall sensor further senses a second magnitude of the magnetic field of the magnet when the magnet is in a vibrated state, and converts the second magnitude into a second voltage value;
wherein the controller is configured to calculate a voltage variation by subtracting the second voltage value from the first voltage value and determines that one step on the walking belt assembly is made by the user and increments the total step number by one when the voltage variation is greater than the predetermined voltage variation threshold.

3. The step-counting treadmill as claimed in claim 1, wherein the controller is configured to calculate calories burned according to the total step number.

4. The step-counting treadmill as claimed in claim 2, wherein the controller is configured to calculate calories burned according to the total step number.

5. The step-counting treadmill as claimed in claim 3, wherein when determining that no step has been sensed for an idle period, the controller is configured to automatically stop running the walking belt and enter a sleep mode, and when determining that any step is sensed during the sleep mode, the controller is configured to restart the walking belt to run and enter a normal mode.

6. The step-counting treadmill as claimed in claim 4, wherein when determining that no step has been sensed for an idle period, the controller is configured to automatically stop running the walking belt and enter a sleep mode, and when determining that any step is sensed during the sleep mode, the controller is configured to restart the walking belt to run and enter a normal mode.

7. A step-counting treadmill comprising:
a frame assembly having:
a lower frame securely mounted on a ground; and
an upper frame mounted on the lower frame;
a dashboard mounted on the upper frame;
a walking belt assembly mounted on the frame assembly;
a controller mounted inside the frame assembly, electrically connected to the dashboard, initializing a left step number, a right step number, a total step number, a step cycle, and an exercise duration of a user in an exercise activity to zero before the exercise activity starts, and starting timing the step cycle and the exercise duration since the exercise activity starts;
a drive motor electrically connected to the controller and instructed by the controller to drive the walking belt assembly to move according to a treadmill speed inputted by the user through the dashboard; and
two step-sensing units electrically connected to the controller, one of the step-sensing units mounted between the walking belt assembly and a left side of the lower frame of the frame assembly, and collaborated with the controller to continuously sense a first voltage variation as a result of a movement change between a vibrated state of the walking belt assembly caused by a movement of the user on the walking belt assembly during the exercise activity and a motionless state of the walking belt assembly before the exercise activity starts, and the other step-sensing unit mounted between the walking belt assembly and a right side of the lower frame of the frame assembly, and collaborated with the controller to continuously sense a second voltage variation as a result of a movement change between a vibrated state of the walking belt assembly caused by another movement of the user on the walking belt assembly during the exercise activity and a motionless state of the walking belt assembly before the exercise activity starts;
wherein when the first voltage variation is greater than a predetermined voltage variation threshold, the controller is configured to determine that one left step on the walking belt assembly is made by the user, increment the left step number by one, set the step cycle as a user's left step cycle, and re-initialize the step cycle to zero, and when the second voltage variation is greater than the predetermined voltage variation threshold, the controller is configured to determine that one right step on the walking belt assembly is made by the user, increment the right step number by one, set the step cycle as a user's right step cycle, and re-initialize the step cycle to zero.

8. The step-counting treadmill as claimed in claim 7, wherein
the walking belt assembly is mounted on the lower frame and includes:
a roller bracket mounted above the lower frame with cushioning pieces sandwiched between the roller bracket and the lower frame; and
a walking belt movably mounted around the roller bracket and driven by the drive motor to move around the roller bracket at the treadmill speed instructed by the controller;
one of the two step-sensing units has:
a first Hall sensor mounted on a left side of the lower frame and electrically connected to the controller; and
a first magnet mounted on the roller bracket to contactlesly correspond to the first Hall sensor in position, wherein the movement of the user exercising on the walking belt assembly causes the first magnet to vibrate for the first Hall sensor collaborated with the controller to continuously sense a change in a magnetic field of the first magnet between the first magnet under a vibrated state and the first magnet under a motionless state and convert the change in the magnetic field of the first magnet into a first voltage variation;
the other step-sensing unit has:
a second Hall sensor mounted on a right side of the lower frame and electrically connected to the controller; and
a second magnet mounted on the roller bracket to contactlessly correspond to the second Hall sensor in position, wherein the movement of the user exercising on the walking belt assembly causes the second magnet to vibrate for the second Hall sensor collaborated with the controller to continuously sense a change in a magnetic field of the second magnet between the second magnet under a vibrated state and the second magnet under a motionless state and convert the change in the magnetic field of the second magnet into a second voltage variation.

9. The step-counting treadmill as claimed in claim 7, wherein the controller is configured to calculate the total step number by adding the left step number and the right step number and use the total step number as one parameter for calculating calories burned.

10. The step-counting treadmill as claimed in claim 8, wherein the controller is configured to calculate the total step number by adding the left step number and the right step number and use the total step number as one parameter for calculating calories burned.

11. The step-counting treadmill as claimed in claim 9, wherein when determining that none of the left step and the right step has been sensed for an idle period, the controller is configured to automatically stop running the walking belt and enter a sleep mode, and when determining that any left step is sensed during the sleep mode, the controller is configured to restart the walking belt to run and enter a normal mode.

12. The step-counting treadmill as claimed in claim 10, wherein when determining that none of the left step and the right step has been sensed for an idle period, the controller is configured to automatically stop running the walking belt and enter a sleep mode, and when determining that any left step is sensed during the sleep mode, the controller restart the walking belt to run and enter a normal mode.

13. The step-counting treadmill as claimed in claim 11, wherein the user inputs a height thereof through the dashboard, the controller is configured to calculate a targeted left step cycle and a targeted right step cycle according to the treadmill speed, the exercise duration of the exercise activity, and the height of the user, and determine that a slow step condition of the user occurs when a difference between the targeted left step cycle and the user's left step cycle at present is greater than a lower targeted gap and is less than an upper targeted gap or a difference between the targeted right step cycle and the user's right step cycle at present is greater than the lower targeted gap and is less than an upper targeted gap.

14. The step-counting treadmill as claimed in claim 12, wherein the user inputs a height thereof through the dashboard, the controller is configured to calculate a targeted left step cycle and a targeted right step cycle according to the treadmill speed, the exercise duration of the exercise activity, and the height of the user, and determine that a slow step condition of the user occurs when a difference between the targeted left step cycle and the user's left step cycle at present is greater than a lower targeted gap and is less than an upper targeted gap or a difference between the targeted right step cycle and the user's right step cycle at present is greater than the lower targeted gap and is less than an upper targeted gap.

15. The step-counting treadmill as claimed in claim 13, wherein the controller is configured to determine that a dangerous step condition of the user occurs when a difference between the targeted left step cycle and the user's left step cycle at present is greater than the upper targeted gap or a difference between the targeted right step cycle and the user's right step cycle at present is greater than the upper targeted gap.

16. The step-counting treadmill as claimed in claim 14, wherein the controller is configured to determine that a dangerous step condition of the user occurs when a difference between the targeted left step cycle and the user's left step cycle at present is greater than the upper targeted gap or a difference between the targeted right step cycle and the user's right step cycle at present is greater than the upper targeted gap.

17. The step-counting treadmill as claimed in claim 15, wherein the controller is configured to determine that an inconsistent step condition of the user occurs when an absolute value of a difference between the user's left step cycle and the user's right step cycle at present exceeds an inter-step cycle variation.

18. The step-counting treadmill as claimed in claim 16, wherein the controller is configured to determine that an inconsistent step condition of the user occurs when an absolute value of a difference between the user's left step cycle and the user's right step cycle at present exceeds an inter-step cycle variation.
